(19) United States
(12) Patent Application Publication
(10) Pub. No.: US 2025/0001087 A1
(43) Pub. Date: Jan. 2, 2025

(54) DRUG DELIVERY DEVICE AND ELECTRONIC UNIT

(71) Applicant: SANOFI, Paris (FR)

(72) Inventors: Stefan Alt, Frankfurt am Main (DE); Tim Glasser, Rüsselsheim (DE); Michael Helmer, Frankfurt am Main (DE); Stephan Mücke, Rüsselsheim (DE); Peter Nober, Rüsselsheim (DE); Michael Schabbach, Frankfurt am Main (DE); Martin Vitt, Frankfurt am Main (DE)

(21) Appl. No.: 18/706,073

(22) PCT Filed: Nov. 1, 2022
(86) PCT No.: PCT/EP2022/080414

§ 371 Date: Apr. 30, 2024

(30) Foreign Application Priority Data
Nov. 3, 2021 (EP) 21315220.0

(51) Int. Cl. A61M 5/315

(52) U.S. Cl. CPC A61M 5/31568; A61M 5/31541; A61M 2205/3317

(57) ABSTRACT

The present disclosure relates to a drive unit of a drug delivery device configured for setting and dispensing of a dose of a medicament, the drive unit comprising: a drive unit housing extending along a longitudinal axis, a drive unit component movably arranged in or on the drive unit housing for at least one of setting of the dose and dispensing of the dose of the medicament, a locking mechanism comprising a mechanical locking element transferable between a release position and a locking position and configured to mechanically engage with the drive unit component when in the locking position to impede at least one of setting of the dose and dispensing of the dose of the medicament, wherein the mechanical locking element is transferable from the locking position into the release position through interaction with an electrically controlled release mechanism of a supplementary device.

### CROSS REFERENCE TO RELATED APPLICATIONS

The present application is the national stage entry of International Patent Application No. PCT/EP2022/080414, filed on Nov. 1, 2022, and claims priority to Application No. EP 21315220.0, filed on Nov. 3, 2021, the disclosures of which are incorporated herein by reference.

### TECHNICAL FIELD

The present disclosure relates to a drive unit of a drug delivery device, in particular to a drive unit of a handheld injection device. The disclosure further relates to a drug delivery system, to a supplementary device for use with the drive unit and the drug delivery device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, e.g. injection devices and needle based injection system (NIS) devices, such as pen-type injectors, have to meet a number of user-specific conditions. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

For example, a patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule can be the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, e.g., the medicament, has been used up.

With reusable injection devices a medicament container, e.g., a cartridge has to be replaced when the medicament has been depleted. Here, the medicament container, e.g. a vitreous cartridge filled with the medicament has to be replaced. Some primary medicament containers, e.g. in form of a cartridge filled with the medicament are preassembled in a housing component of the injection device, such as a cartridge holder.

There further exist electronic units, e.g. either integrated into such delivery or injection devices or provided as a separate device, also denoted as a supplementary device or add-on device. Such supplementary devices provide additional functionality to an all-mechanically implemented drug delivery or injection device. By way of a supplementary device, repeated setting and dispensing or injecting of doses of the medicament can be precisely monitored and logged over time. Supplementary devices offer a large variety of supplemental functions for the regular use of drug delivery or injection devices. Supplementary devices may offer data analysis and further communication of such data to health care providers. Such data being indicative about the amount of a medicament administered at a particular point of time or at a particular date.

By way of electronically implemented supplementary devices, e.g. detachably mechanically connectable to a drug delivery or injection device handling and operation of such drug delivery devices can be facilitated.

In view of the above it is a further aim to enhance patient safety and to increase the functional scope of such supplementary devices, of drug delivery devices for use with such supplementary devices and to provide an improved injection system comprising a drug delivery device and a supplementary device.

### SUMMARY

In one aspect the present disclosure relates to a drive unit of a drug delivery device. The drug delivery device may be implemented as an injection device, e.g. as a handheld injection device, in particular as a pen-type injection device. The drug delivery device and hence the drive unit is configured for setting and dispensing of a dose of a medicament. The drive unit comprises a drive unit housing extending along a longitudinal axis. The drive unit further comprises a drive unit component, i.e. a mechanical component, movably arranged in or on the drive unit housing for at least one of setting of the dose and dispensing of the dose of the medicament.

The drive unit further comprises a locking mechanism. The locking mechanism comprises a mechanical locking element. The mechanical locking element is transferable between a release position and a locking position. It is hence transferable between a release configuration and a locking configuration. It is configured to mechanically engage with the drive unit component when it is in the locking position or locking configuration. When the mechanical locking element is in the locking position it is operable to impede at least one of setting of the dose and dispensing of the dose of the medicament. When in the locking position or locking configuration the mechanical locking element is in mechanical engagement with the movable drive unit component and blocks and/or impedes a respective movement thereof.

The mechanical locking element is exclusively or only transferable from the locking position into the release position through interaction with an electrically controlled release mechanism of a supplementary device. The supplementary device is configured for attachment to the drive unit housing. Typically, the drive unit housing is mechanically engageable with the supplementary device. The supplementary device may be detachably connectable or detachably fixable to the drive unit housing. When appropriately fixed or attached to the drive unit housing the release mechanism of the supplementary device is operable to unlock the mechanical locking mechanism of the drive unit.

The locking mechanism of the drive unit is particularly implemented to be only and exclusively transferable into a release or unlocked configuration through interaction with the electrically controlled release mechanism of the supplementary device.

With some examples it is intended that the locking mechanism is in its locking position per default. Then and for unlocking the drive unit, the release mechanism of the supplementary device is used. This may, for example, require a proper and correct assembly of the supplementary device to the drive unit housing of the drive unit. The absence or an incorrect mounting or attaching of the supplementary device to the drive unit housing may lead to an improper arrangement or alignment of the release mechanism relative to the locking mechanism. Accordingly and when the supplementary device is not correctly attached to the drive unit housing the release mechanism of the supplementary device will not be able and will be hence inoperable to transfer the locking mechanism of the drive unit into the release configuration or release position.

In this way, a correct and proper attachment of the supplementary device to the drive unit or drive unit housing becomes a prerequisite to unlock the drive unit either for setting of the dose and/or for dispensing or injecting of the dose of the medicament.

With some examples, the mechanical locking element of the drive unit of the drug delivery device is directly movable or transferable by the release mechanism of the supplementary device. This way, the drive unit of the drug delivery device can be void of any electrically or electronically implemented actuators by way of which the mechanical locking element could be transferred from the locking position into the release position. Such electromechanically implemented actuators may be exclusively provided by the release mechanism of the supplementary device. This solution may be rather cost efficient because the supplementary device is intended for a long-term use with numerous drug delivery devices, whereas a drive unit of a drug delivery device may be part of a disposable drug delivery device or disposable injection device.

With some examples the mechanical locking element is only transferable or movable by an actuator of the release mechanism of the supplementary device. This way, the drive unit of the drug delivery device can be implemented without an electromechanically implemented actuator. Hence, the drug delivery device and the drive unit may be void of electromechanically implemented components thus allowing a rather cost-efficient design and implementation of the drug delivery device.

Unlocking of the drive unit of the drug delivery device exclusively and only by way of a supplementary device may be beneficial in many aspects. In one aspect unlocking of the drive unit may be only possible when the supplementary device is correctly attached or assembled to the drive unit housing. Hence, the drive unit and the drug delivery device cannot be used without the presence and/or without a correct installation of the supplementary device.

In a further aspect the supplementary device itself may be provided with an electrically or electronically implemented authentication function or feature by way of which authorized access to the supplementary device and hence authorized access to the drive unit can be electronically controlled. Hence, the supplementary device may be implemented to transfer the mechanical locking element of the drive unit only then into the release position or release configuration when a user intending to use the drive unit or drug delivery device has successfully passed an authentication procedure with the supplementary device.

Moreover, the supplementary device may be program-controlled and may provide a kind of an automated locking or unlocking of the drive unit. With some examples the supplementary device may be provided with patient specific data, e.g. being indicative of at least one of an amount of a medicament and a time or date when an injection is due. A user attempting to use the drive unit at any other time to administer a dose of inappropriate size may be effectively hindered to do so because the supplementary device may be configured to unlock the locking mechanism only when an actuation or operation of the drive unit as induced by the user matches with a predefined prescription or medication schedule.

With further examples the authentication function conducted by the supplementary device may be operable to distinguish between different container units, e.g. equipped with different drug containers or medicament. Here, the drive unit may be configured to engage with a number of differently configured container units, e.g. that differ by the amount, the concentration and by the type of a drug or medicament. Each one of the container units being generally attachable to the drive unit may be provided with a unique identifier, e.g. by an electrically detectable or visual code or coding feature. Here, the supplementary device may be operable to read or to extract the relevant code information so as to obtain respective information about the drug or medicament provided in the container unit.

The container unit equipped with a medicament container may be visually or electronically encoded by a respective coding feature, e.g. implemented as a barcode, as a QR code or in form of an electronically readable code, e.g. provided by RFID chip. The supplementary device and/or an external electronic device operable to communicate with the supplementary device may be operable to capture and/or to read or to extract the coding feature of the container unit. Based on the captured coding feature at least one of the supplementary device and the external electronic device may evaluate the coding feature. The evaluation of the coding feature may include a comparison of the coding feature with user specific information, e.g. stored in one of the supplementary device and the external electronic device. If the coding feature matches with the user specific information, e.g. if it should be determined, that the type of medicament located inside the container unit matches a prescription schedule of a particular user, the locking mechanism should be transferred into the release configuration so as to allow and to support administering of a dose of the medicament.

In other cases and if it should be determined that the type of medicament does not match e.g. with a predefined prescription schedule of a particular user, the electronic unit, hence its processor is operable to keep the locking mechanism in a locked configuration so as to protect the patient against administering of a medicament that does not match with his prescription schedule. This way, misuse or unintended use of the drive unit can be then effectively prevented.

Authentication to switch the locking mechanism into the release configuration may not be limited to the detection of a correct medicament but may also include compliance with a predefined time schedule. For instance, and if a user should attempt to administer a medicament at a time that does not match with a predefined prescription schedule the locking mechanism may also remain in the interlock configuration.

According to a further example the mechanical locking element is movably arranged inside the drive unit housing. The mechanical locking element may be located inside an opaque portion of the drive unit housing. It may be concealed by the drive unit housing and may be arranged in a visually hidden section of the drive unit housing. Hence, it may not be apparent from outside the drive unit or drive unit housing, where the mechanical locking element is actually located. Nevertheless, the release mechanism of the supplementary device is enabled and configured to mechanically interact with the mechanical locking element when the supplementary device is correctly attached, fixed or assembled to the drive unit housing.

Typically, the mechanical locking element is located inside the drive unit housing in its entirety. It does not protrude from the drive unit housing and does not intersect the drive unit housing. This way, the mechanical locking element remains totally hidden and covered inside the drive unit housing. Such an arrangement is of particular use to prevent unauthorized movement or reconfiguration of the mechanical locking element, i.e. to reconfigure or to rearrange the mechanical locking element without use of the supplementary device.

According to a further example the mechanical locking element is mechanically inaccessible from outside the drive unit housing. Hence, the drive unit housing may entirely encapsulate the mechanical locking element. Movement or reconfiguration of the mechanical locking element may then, for example, require some other type of interaction, e.g. an electrically induced interaction between the release mechanism and the mechanical locking element. Such interaction may be based on magnetic forces or electrostatic forces transferable through the drive unit housing.

Moving or reconfiguring the mechanical locking element to transfer the mechanical locking element from the locking position towards and into the release position may, for example, require the presence of dedicated magnetic or electrostatic forces at a particular position on an outside surface of the drive unit housing, which forces are generally difficult to generate without the help of a suitable supplementary device.

With other examples the drive unit housing may comprise a recess or an access opening in line with the mechanical locking element, such that a dedicated portion, e.g. in form of a movable pin of the release mechanism of the supplementary device may access the mechanical locking element by entering the recess and/or by penetrating or extending through the access opening of the drive unit housing.

With such an example it is intended that the recess and/or the access opening comprises a diameter or a cross-section that is smaller, even much smaller than the diameter or finger of a user. Moreover, such a recess or access opening may comprise a rather tiny and small cross-sections requiring, for example, a special tool, such as a dedicated pin of the supplementary device to successfully enter the recess or access opening.

With further examples, the recess or access opening in line with the mechanical locking element is provided with a keyed structure, such as a regular or irregular cross-sectional geometric structure which can only receive a complementary shaped pin of a supplementary device.

According to a further example the mechanical locking element comprises a body with at least one of a ferromagnetic material and a magnetized material to magnetically interact with the release mechanism. Here, the release mechanism of the supplementary device is typically provided with an actuator comprising an electromagnet. The actuator of the supplementary device is configured to generate a magnetic field interacting with the ferromagnetic material and/or with the magnetized material of the mechanical locking element so as to transfer the mechanical locking element from one of the locking position or locking configuration towards and into the release position or unlocking configuration.

Such a magnetic interaction is of particular benefit for that the mechanical locking element can be entirely encapsulated inside the drive unit housing.

According to a further example the drive unit housing comprises a fastening structure configured to detachably engage with a complementary shaped fastener of the supplementary device. The fastening structure is arranged on or in the drive unit housing such that upon engagement with the complementary shaped fastener of the supplementary device the mechanical locking element is aligned with an actuator of the release mechanism of the supplementary device. Accordingly, a geometric alignment between the actuator of the release mechanism and the mechanical locking element of the locking mechanism is obtained when the fastening structure is correctly engaged with the complementary shaped fastener of the supplementary device.

Typically and with further examples, the mechanical locking element is misaligned or is not in alignment with the actuator of the release mechanism when the fastening structure is incorrectly engaged or is not engaged with the complementary shaped fastener of the supplementary device. With a misaligned or non-aligned arrangement of the mechanical locking element and the actuator the actuator is inoperable to transfer the mechanical locking element from the locking position towards or into the unlocking position or release position. This way and for unlocking of the locking mechanism, assembly of the supplementary device to or on the drive unit housing of the drive unit should be correct.

With a further example the mechanical locking element is movably fastened to one of the drive unit component and the drive unit housing. The mechanical locking element is configured to engage with the other one of the drive unit component and the drive unit housing when transferred from the release position into the locking position. When the mechanical locking element is movably fastened, e.g. to the drive unit housing, it is out of engagement with the drive unit component as long as it is in the release position or release configuration. Transferring the mechanical locking element into the locking position brings the mechanical locking element into engagement with the drive unit component.

In the locking configuration or blocking position the mechanical locking element may be directly or indirectly mechanically engaged with the drive unit component and the drive unit housing thereby blocking or impeding a movement of the drive unit component relative to the drive unit housing. In effect and when the mechanical locking element is in the locking position the drive unit component is blocked from moving relative to the drive unit housing. Here and when in the locking configuration setting of a dose or dispensing of a dose is effectively blocked due to the positional fixing of the drive unit component relative to the drive unit housing.

With other examples the mechanical locking element is movably fastened on or in the drive unit housing. Transferring the mechanical locking element into the locking position or locking configuration brings the mechanical locking element into mechanical engagement with the drive unit component thereby blocking a movement of the drive unit component relative to the drive unit housing.

When engaged with the drive unit housing and with the drive unit component the mechanical locking element is operable to prevent at least one of a longitudinal movement and a rotational movement of the drive unit component relative to the drive unit housing. With further examples, the drive unit component may protrude from the drive unit housing. It may form a component of an outer shell of the drive unit. Here, and when the locking mechanism gets into the locking position it may be operable to lock the drive unit component relative to a second drive unit component, wherein the second drive unit component is at least temporally movably fixed to the drive unit housing. In this way, a kind of an indirect mechanical fixing of the drive unit component relative to the drive unit housing can be obtained by the mechanical locking element.

With a further example the drive unit component is rotatable with regard to the longitudinal axis for at least one of setting of the dose and dispensing of the dose. The drive unit component comprises a tubular-shaped sidewall with a toothed structure. The locking element comprises a latch movable, pivotable or deformable in a radial direction to engage with the toothed structure. Typically, the latch comprises a counter toothed structure to mechanically engage with the toothed structure of the sidewall of the drive unit component. In this way and when the latch engages with the toothed structure of the drive unit component the drive unit component is hindered to rotate any further with regards to the longitudinal axis as an axis of rotation.

Typically, at least one of the latch and the toothed structure comprises a protrusion to engage with a complementary-shaped recess provided on the other one of the latch and the toothed structure. With some examples the toothed structure is provided on an outside facing sidewall of the tubular shaped drive unit component. Then, the latch may be biased radially inwardly in order to engage with the toothed structure. With other examples the toothed structure is provided on an inside surface of the sidewall of the drive unit component. Then, the latch is typically biased radially outwardly to engage with the toothed structure.

Interaction between the release mechanism of the supplementary device and the mechanical locking element is typically implemented such that the engagement between the latch and the toothed structure can be at least temporally abrogated, e.g. when the electrically controlled release mechanism of the supplementary device is actuated or activated. With some examples the drive unit component is implemented as a number sleeve, as a rotatable clutch element, or as a drive sleeve of the drive unit configured to rotate relative to the drive unit housing for at least one of setting of the dose and dispensing of the dose.

According to a further example the drive unit component is longitudinally displaceable for at least one of setting of the dose and dispensing of the dose. The locking mechanism comprises a retaining element longitudinally secured to the drive unit component with regard to the longitudinal axis. The retaining element is movable in radial direction with regard to the longitudinal axis between a retaining position and a release position. With some examples and when in the retaining position the retaining element may radially outwardly or radially inwardly protrude from the drive unit component and may axially or longitudinally engage with a further drive unit component or with the drive unit housing.

This longitudinal engagement may serve to block or to impede a longitudinal movement of the retaining member and hence a longitudinal movement of the drive unit component relative to the further drive unit component and/or relative to the drive unit housing. Likewise, with the radially protruding retaining element there may be additionally or alternatively blocked a rotational movement of the drive unit component relative to the drive unit housing and/or relative to a further drive unit component.

With some examples, there may be provided two or more retaining elements each of which being secured to the drive unit component with regards to the longitudinal axis and being movable in radial direction relative to the drive unit component. In this way, there can be provided a twofold or even threefold, hence a multiple longitudinal abutment between the drive unit component, the retaining element and at least one of a further drive unit component and the drive unit housing. Here, the further drive unit component is longitudinally and/or rotationally secured to the housing.

In effect and when the retaining element protrudes radially from e.g. a sidewall of the tubular shaped drive unit component the drive unit component is effectively hindered to be moved in longitudinal direction, hence along or parallel to the longitudinal axis.

With a further example the locking element is movably supported in or on the drive unit component and is mechanically engaged with the retaining element. A longitudinal movement of the locking element from the locking position towards the release position is effective to cause a movement of the retaining element from the retaining position towards the release position. Moreover, the mutual mechanical engagement between the drive unit component, the locking element and the retaining element(s) may be such that a longitudinal movement of the locking element from the release position towards the locking position is effective to cause a movement of the retaining element from the release position towards and into the retaining position.

In this way, the longitudinal movement of the locking element relative to the drive unit component serves to induce a radially directed movement of the at least one retaining element by way of which the retaining element longitudinally engages with at least one of the drive unit housing and a further drive unit component longitudinally secured to the drive unit housing. In this way there can be provided a mechanical interlock for the drive unit component. The radially directed movement of the retaining element may either be operable to block a longitudinal displacement of the drive unit component relative to the drive unit housing and/or to block a longitudinal sliding movement between the drive unit component and the drive unit housing either directly or indirectly, e.g. via another drive unit component longitudinally and/or rotationally secured to the drive unit housing.

With another example the drive unit component is movable from an initial position towards an end position for at least one of setting of the dose and dispensing of the dose and wherein when in the locking position the mechanical locking element is operable to fix the drive unit component in the initial position. Here, and when transferred or switched onto the release position the mechanical locking element releases the drive unit component and enables or allows a movement of the drive unit component towards the end position.

Typically, and when the drive unit component is implemented as one of a dose dial and a number sleeve, which is movable for setting of a dose the mechanical locking element is configured to lock the device component in the initial position which may correspond to a so-called zero-dose configuration. Then, the drive unit may be locked in the zero-dose configuration. Hence, setting of a dose may be effectively blocked.

With other examples and when the drive unit component is implemented as a trigger or dose button depressible for dispensing of the dose the mechanical locking element is configured to impede and/or to block a dose dispensing action.

With still another example the drive unit component comprises an elongated bore in which the mechanical locking element is slidably disposed or slidably arranged. Here, the mechanical locking element is slidably displaceable relative to the drive unit component between the release position and the locking position. Typically, the mechanical locking element may be slidably disposed inside the elongated bore between a distal end position and a proximal end position. In the distal end position the mechanical locking element may be in the release position or release configuration. In the proximal end position the mechanical locking element may be in the blocking position. With other examples the release position coincides with a proximal end position and a locking position coincides with a distal end position. Typically, the elongated bore extends parallel to the longitudinal axis.

The elongated bore may be open towards a proximal end so as to receive a complementary-shaped pin of the electrically controlled release mechanism of the supplementary device when the supplementary device is appropriately mounted on the drive unit housing. The diameter or cross-section of the elongated bore typically matches with a complementary diameter or cross-section of a pin of the release mechanism of the supplementary device. It is configured to enter the elongated bore when the supplementary device is correctly assembled and mounted on the drive unit housing.

When correctly assembled or mounted on the drive unit housing a movable pin of the electrically controlled release mechanism is operable to enter the elongated bore in longitudinal direction so as to induce a longitudinal sliding displacement of the mechanical locking element inside the bore.

With a further example the locking element comprises an elongated locking bolt. The locking bolt comprises a radial recess sized to receive at least a portion of the retaining element. With some examples the elongated locking bolt comprises a radial groove extending around the locking bolt. Hence, the radial recess comprises an annular shape extending along the outside circumference of the locking bolt. It may be configured to receive a plurality of retaining elements.

As seen in longitudinal direction of the elongated locking bolt the radial recess may comprise a beveled section. Hence, the radial recess may comprise a kind of a V-shaped groove as seen in longitudinal direction. In this way and as the locking bolt or the locking element is subject to a longitudinal displacement inside the elongated bore the retaining element is pushed in radial direction as it engages with the beveled section of the radial recess.

In this way a longitudinally directed motion of the elongated locking bolt or locking element relative to the elongated bore can be transferred into a radially directed movement of the retaining element. With some examples the retaining element comprises a number of retaining balls. Here, the single retaining elements may comprise a spherical shape or geometry. A diameter of the retaining element(s) may be larger than a thickness of the sidewall of the drive unit component. In this way and as the retaining elements engage with a slanted or beveled edge of the radial recess of the elongated locking bolt at least a portion of the retaining element will be urged radially outwardly and tend to protrude radially outwardly from a sidewall of the drive unit component, thereby bringing the drive unit component into a locking engagement either with the drive unit housing or with a further drive unit component connected or fixed to the housing.

According to a further example the drive unit component comprises a sidewall with a through recess in which the retaining element is axially retained and in which the retaining element is movable in radial direction. In this way and as the retaining element(s) engage(s) with the slanted or beveled edge of the radial recess of the elongated locking bolt the retaining element(s) is hindered to move in longitudinal direction. The longitudinal motion of the mechanical locking element relative to the drive unit component is then entirely transferred into a radial movement of the retaining element(s).

With some examples the retaining element may comprise numerous retaining segments, each of which being longitudinally fixed to the drive unit component or to the sidewall of the drive unit component. Here, each one of retaining segments or retaining elements might be individually movable in radial direction relative to the drive unit component.

The retaining element may be implemented as an annularly segmented structure allowing and supporting a radially outwardly directed extension against an intrinsic radially inwardly directed restoring force. Here, the retaining element could be made of an elastic material, configured to elastically transfer from the retaining position towards and back into the release position in the absence of a force effect acting on the elongated locking bolt. Hence, the retaining element or the plurality of retaining elements may be operable to relax or to return into the retaining position thereby providing an automated activation of the locking mechanism.

According to a further example the locking mechanism comprises a return spring engaged with the mechanical locking element and operable to transfer the mechanical locking element from the release position into the locking position. The return spring may be configured to move or to pivot the mechanical locking element from the release position into the locking position. In this way the locking mechanism automatically interlocks, e.g. in the absence of the electrically controlled release mechanism of the supplementary device. With other examples the mechanical locking element itself is provided with a return spring or is implemented as an elastically deformable mechanical locking element. Here, at least one of the material and the geometric shape of the mechanical locking element serves to provide a return force by way of which the mechanical locking element remains or returns into an initial or unbiased shape. Also here, a rather automated activation of the locking mechanism can be provided.

Typically, the drive unit component is a movable component of the drive unit. It may be implemented as one of a number sleeve, a dial sleeve, a drive sleeve, as a clutch element or as any other movable component of the drive mechanism that is subject to at least one of a rotational or longitudinal movement for setting of the dose order or for dispensing of the dose.

According to another aspect the present disclosure relates to a drug delivery device for setting and dispensing of a dose of a medicament. The drug delivery device comprises a drive unit as described above. The drug delivery device further comprises a container unit configured to accommodate a medicament container and being further configured to fasten to the drive unit housing. The drive unit is configured to operably engage with the medicament container in order to withdraw or to expel the dose of the medicament from the medicament container. With some examples the drug delivery device comprises an injection device. The drug delivery device may be implemented as a handheld injection device, such as an injection pen. With some examples the drive unit comprises an all-mechanically implemented drive unit, wherein a user of the drug delivery device has to provide a dispensing force effective for withdrawing or for expelling the dose of the medicament from the medicament container.

With some examples the medicament container comprises a cartridge extending along the longitudinal axis and being sealed towards a proximal end by a movable stopper. Here, the drive unit comprises an elongated piston rod or plunger to exert distally directed pressure onto the stopper. This way a pressure inside the medicament container can be increased so as to expel a dose of the medicament via a distal outlet, which is typically in fluid connection with an injection needle. Thus, the piston rod is operable to urge the stopper in distal direction relative to a barrel of the cartridge or medicament container to expel the dose of the medicament.

According to another example the drug delivery device comprises the medicament container arranged inside the container unit. Typically, the container unit comprises a container holder, e.g. implemented as a cartridge holder sized and configured to accommodate the medicament container. The container unit may comprise the medicament container implemented as a cartridge and may comprise a cartridge holder sized and shaped to accommodate and to fix the cartridge therein. The cartridge holder and hence the container holder of the container unit is configured to fasten and to fix with the drive unit housing so as to form or to constitute the housing of the drug delivery device. The container unit may be detachably or non-detachably fastened or fixed to the drive unit housing. With a reusable drug delivery device the drive unit housing and the container unit are detachably fixed. With a disposable drug delivery device the drive unit housing is typically non-detachably fixed to the container unit.

Generally and since the drive unit as described above is a component of the drug delivery device, features, effects and benefits as described above in connection with the drive unit equally apply to the drug delivery device; and vice versa.

According to another aspect the present disclosure relates to a supplementary device for use with a drive unit as described above. The supplementary device comprises a housing configured for attachment to the drive unit as described above. The supplementary device further comprises an electronic unit arranged inside the housing. The supplementary device further comprises a release mechanism comprising an electromagnetic actuator electrically controlled by the electronic unit and operable to transfer the mechanical locking element of the locking mechanism of the drive unit into the release position. The electromagnetic actuator may be configured to directly or to indirectly engage with the mechanical locking element of the locking mechanism of the drive unit. Since the supplementary device is configured for use with a drive unit as described above, features, effects and benefits as described above in connection with the drive unit equally apply to the supplementary device; and vice versa.

With another example the electromagnetic actuator of the supplementary device comprises one of an electromagnet and a movable pin. The electromagnet is operable to magnetically interact with the mechanical locking element, especially when the mechanical locking element of the locking mechanism of the drive unit comprises at least one of a ferromagnetic material and a magnetized material. In this way and upon activation of the electromagnet there will be generated a magnetic field operable to move or to pivot the mechanical locking element from the locking position towards and into the release position; or vice versa, to move or to pivot the mechanical locking element from the release position towards and into the locking position.

With other examples the electromagnetic actuator comprises a movable pin to mechanically engage with the mechanical locking element. A movable pin may be particularly configured to push or to drag the mechanical locking element when slidably disposed in an elongated bore of the drive unit component.

Typically, the movable pin may be sized and shaped to enter the elongated bore of the drive unit component when the supplementary device is appropriately connected and fixed to the drive unit housing.

In still another aspect the present disclosure relates to a drug delivery system. The drug delivery system may be implemented as an injection system. It comprises a supplementary device as described above and further comprises a drive unit and/or a drug delivery device as described above. Insofar, features, effects and benefits as described above in connection with the supplementary device, the drive unit and/or as described in connection with the drug delivery device equally apply to the drug delivery system; and vice versa.

Generally, the scope of the present disclosure is defined by the content. The injection device is not limited to specific embodiments or examples but comprises any combination of elements of different embodiments or examples. Insofar, the present disclosure covers any combination of embodiments and any technically feasible combination of the features disclosed in connection with different examples or embodiments.

It will be further apparent to those skilled in the art that various modifications and variations can be made to the present disclosure without departing from the scope of the disclosure.

In the present context the term ‘distal’ or ‘distal end’ relates to an end of the drug delivery device or injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des (B30) human insulin, Lys(B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N—(ω-carboxyheptadecanoyl)-des (B30) human insulin and B29-N—(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

### BRIEF DESCRIPTION OF THE DRAWINGS

In the following, numerous examples of a drive unit of a drug delivery device configured to interact with a supplementary device, of a drug delivery device, of a supplementary device and of a drug delivery system are illustrated and described in greater detail by making reference to the drawings, in which:

FIG. 1 illustrates one example of a drug delivery device implemented as a handheld pen-type injector.

FIG. 2 shows an enlarged longitudinal cross-section through the drive unit of the drug delivery device according to FIG. 1.

FIG. 3 shows a cross-section through the mechanical locking element in the locking position.

FIG. 4 shows the mechanical locking element in the release position,

FIG. 5 illustrates a perspective view of the supplementary device before attached to the drive unit housing.

FIG. 6 shows details of a fastening mechanism for fastening and fixing the container unit to the drive unit, wherein the fastening mechanism is in an interlocked configuration.

FIG. 7 shows the fastening mechanism of FIG. 6 when in a release configuration,

FIG. 8 shows a block diagram of the drug delivery system.

FIG. 9 illustrates another drive unit of another drug delivery device.

FIG. 10 shows an enlarged cross-sectional view of the drive unit of the drug delivery device of FIG. 9 with a locking mechanism in the locking position or locking configuration.

FIG. 11 shows the locking mechanism of FIG. 9 in the release position or release configuration.

### DETAILED DESCRIPTION

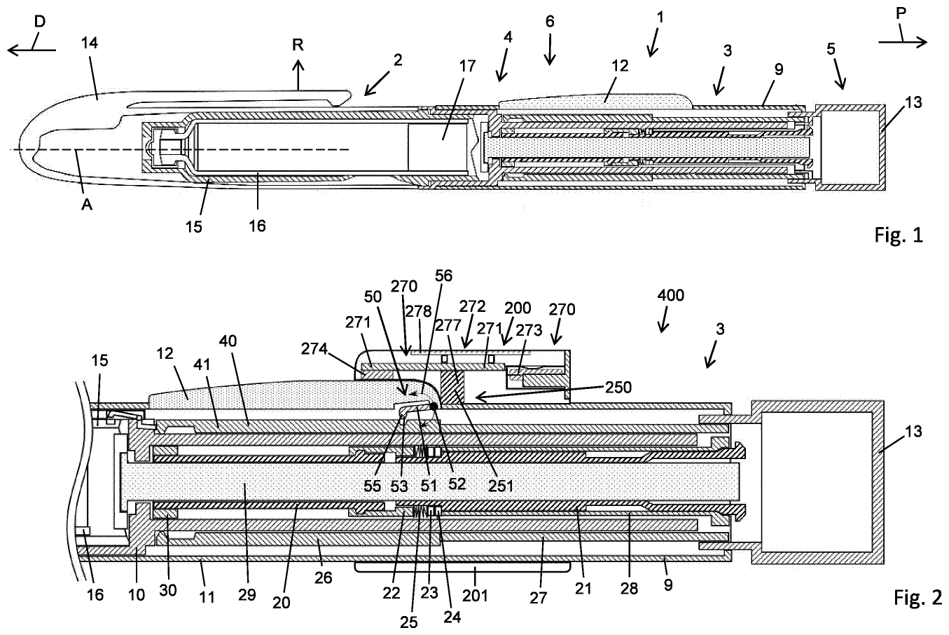

FIGS. 1 and 2, and show an exemplary embodiment of a drug delivery device 1 in a cross-sectional view. The drug delivery device 1 is a variable dose device, in which different doses of a drug to be dispensed can be set or dialed, respectively, by a user. The drug delivery device is a dial extension pen.

FIG. 1 also indicates the coordinate system used herein for specifying positions of members or elements or features. The distal direction D and proximal direction P run parallel to the longitudinal axis A. The longitudinal axis A is a main extension axis of the device 1. The radial direction R is a direction perpendicular to the longitudinal axis A and intersecting with the longitudinal axis A. A tangential direction, also referred to as circumferential direction is a direction perpendicular to the radial direction R and to the longitudinal axis A.

The drug delivery device 1 comprises a drive unit 3 with a setting mechanism and a dispense mechanism. The setting mechanism is configured for setting a drug dose and the dispense mechanism is configured for dispensing a drug dose. The functional principles of the mechanisms are explained further below.

The drive unit 3 comprises an inner body 10 and a housing element 11, in the following also referred to as outer body 11. The inner body 10 and the outer body 11 are fixedly connected to each other, e.g., they cannot be rotated or moved axially with respect to each other. The inner body 10 and outer body 11 may form or constitute a drive unit housing 9. The outer body 11 forms an outer surface of the drug delivery device 1 which can be touched or grabbed by a user. The drive unit 3, hence the drive unit housing 9 comprises a distal end 4 configured for connection with a proximal end of a container unit 2. the drive unit 3 further comprises a proximal end 5, provided with the dose button 13. Hence, the dose button 13 may form or constitute the proximal end 5 of the drive unit housing 9.

The drug delivery device 1 further comprises a protective cap 14 and a user interface member 13 in form of a knob 13. The knob 13 is a dose setting member configured to be operated by a user for setting a drug dose. At the same time, the knob 13 is dose dispense member configured to be operated by a user in order to dispense a drug dose.

A drug reservoir unit 2, also referred to as a container unit 2, comprises a reservoir 16 and a reservoir holder 15 is received within the cap 14. The reservoir 16 is filled with a drug or medicament. The reservoir 16 is sealed in proximal direction P by a stopper 17.

The reservoir unit 2 is operatively coupled or connected, respectively, to the drive unit 3. The drive unit 3 is configured to enable a dispense process for dispensing a drug dose by acting on the drug reservoir 16. For dispensing a drug dose, the stopper 17 is pushed in distal direction D by a plunger rod 29 of the drive unit 3. When an injection needle 18 is connected to drug reservoir a dose of the medicament can be injected into biological tissue.

The coupling between the drive unit 3 and the reservoir unit 2 is realized by the inner body 10 being coupled to the reservoir holder 15 via a connection interface which might be a snap connection or a threaded connection. The coupling can be reversible. For example, the container unit 2 is axially and rotationally fixed to the inner body 10 by the coupling.

The drive unit 3 further comprises a number sleeve 26 and a dial sleeve 27 which are fixedly coupled to each other (e.g. they cannot rotate or move axially relative to each other). The dial sleeve 27 may comprise an inner thread which is engaged with an outer thread of the inner body 10. On an outer surface of the number sleeve 26, numbers may be shown. The user can see the numbers through a window 12. The window 12 may comprise a lens. The window 12 is formed in the outer body 11. The numbers visible in the window 12 indicate to a user the set/dialed dose. Due to the threaded coupling between the dial sleeve 27 and the inner body 10, the dial sleeve 27 and the number sleeve 26 are moved on a helical path in proximal direction relative to the body 10, 11 during setting a drug dose and dispensing a drug dose as will be explained further below.

The drive unit 3 also comprises a drive sleeve. The drive sleeve comprises a distal drive sleeve 20, a proximal drive sleeve 21 and a drive sleeve coupler 22 coupling the distal drive sleeve 20 to the proximal drive sleeve 21. For setting a drug dose and dispensing a drug dose, the distal drive sleeve 20 and the proximal drive sleeve 21 are fixedly coupled to each other via the drive sleeve coupler 22 so that these elements can neither rotated nor move axially relative to each other. The distal drive sleeve 20 may comprise an inner thread which is engaged with an outer thread of the plunger rod 29. An outer thread of the distal drive sleeve 20 may be engaged to an inner thread of a last dose nut 30, the function of which will be explained further below.

Furthermore, the drive unit 3 comprises a clutch 28, which is fixedly coupled to the knob 13 so that, during setting a drug dose and dispensing a drug dose, the clutch 28 and the knob 13 are not rotated or moved axially relative to each other. The clutch 28 is coupled to the proximal drive sleeve 20 via a splined engagement. This splined engagement may allow a certain axial movement of the clutch 28 relative to the proximal drive sleeve 21 but does not allow a relative rotation between these two elements.

A distal clicker 23, a proximal clicker 24 and a clutch spring 25 of the drive unit 3 are arranged between the clutch 28 and the drive sleeve coupler 22. The clutch spring 25 is coupled to the drive sleeve coupler 22 and to the distal clicker 23. The distal clicker 23 is configured to abut against the proximal clicker 24 in proximal direction P. The proximal clicker 24 is configured to abut against the clutch 28 in proximal direction P. Thus, the clutch spring 25 is configured to bias the distal clicker 23, the proximal clicker 24 and the clutch 28 in proximal direction P relative to the drive sleeve coupler 22.

The distal clicker 23 may be permanently splined to the proximal drive sleeve 21 so that a relative rotation between these two elements is prevented. However, a certain axial movement between the distal clicker 23 and the proximal drive sleeve 21 may be allowed. The proximal clicker 24 may be permanently splined to the inner body 10 so that a relative rotation between these two elements is prevented, whereas a certain relative axial movement may be allowed.

The distal face of the clutch 28 and the proximal face of the proximal clicker 24 may both be toothed so that these two faces may engage into each other. Furthermore, the distal face of the proximal clicker 24 and the proximal face of the distal clicker 23 may both be toothed so that these two toothed faces can engage into each other. A proximal face of the clutch 28 may be toothed, e.g. dog toothed, and may be arranged to engage a toothed, e.g. dog toothed, distal face of the dial sleeve 27.

FIG. 1 shows the drug delivery device 1 when no dose is set (0 units/0 unit position). Dose setting may be allowed in discrete units of 1, e.g. from 0 to 80 units. For setting a desired drug dose, the user has to rotate the knob 13. This is done without pressing on the knob 13 in distal direction D. As long as one does not press on the knob 13 in distal direction D, a dog toothed engagement between the clutch 28 and the dial sleeve 27 is established due to the clutch spring 25 either biasing the clutch 28 in proximal direction P or at least preventing the clutch 28 from moving in distal direction D on its own. The dog toothed engagement between the clutch 28 and the dial sleeve 27 has as a consequence that the two elements are rotationally locked to each other so that, when the knob 13 is rotated, also the dial sleeve 27 and the number sleeve 26 are rotated. Since the dial sleeve 27 is threadedly engaged with the inner body 10, rotating the knob 13 has as a consequence that the knob 13, the clutch 28, the dial sleeve 27 and the number sleeve 26 move on a helical path in proximal direction P relative to the body 10, 11. Thereby, the numbers of the number sleeve 26 visible through the window 12 increase.

Since the proximal drive sleeve 21 is splined to the clutch 28, also the proximal drive sleeve 21 and with it the distal drive sleeve 20 and the drive sleeve coupler 22 are moved on the helical path in proximal direction P relative to the inner body 10.

The plunger rod 29 comprises two outer threads with opposite hand which overlap with each other. The plunger rod 29 is threadedly engaged with the inner thread of the distal drive sleeve 20. The threads are chosen such that during the helical movement of the distal drive sleeve 20 in proximal direction P, the plunger rod 29 does not rotate and is also not moved axially.

The last dose nut 30 may be splined to the inner body 10 and, therefore, cannot rotate relative to the inner body 10. Due to the threaded engagement of the last dose nut 30 with the distal drive sleeve 20, the last dose nut 30 is forced to move in proximal direction P during setting a drug dose. When the maximum dose has been set (e.g. 80 units-independently of whether it has been set in only one drug setting process or several drug setting processes), the last dose nut 30 establishes a rotation-lock interface with the distal drive sleeve 20 so that the last dose nut 30 can no longer rotate relative to the distal drive sleeve 20. As a consequence of this, the distal drive sleeve 20 can no longer be rotated and no further drug dose can be set. The drug delivery device 1 then has to be reset to its initial state.

During setting a drug dose, the toothed faces of the distal clicker 23 and the proximal clicker 24 facing each other ratchet over each other thereby creating a click sound which indicates to a user that a drug dose is set. For this purpose, the teeth of the two faces can be formed as shallow triangles so that relative rotation between the clickers 23 and 24 is possible leading to a repeated slight compression and decompression of the clutch spring 25.

After the desired dose has been set, the user can now press on the knob 13 in distal direction D in order to dispense the set drug dose. Thereby, the distally directed force on the knob 13 is transferred from the knob 13 via the clutch 28 to the proximal clicker 24, from there to the distal clicker 23 and this compresses the clutch spring 25. The two clickers 23 and 24 are now pressed against each other and their toothed faces are engaged. Relative rotation between the two clickers 23, 24 is now prevented. Since the proximal clicker 24 is splined to the inner body 10 and the distal clicker 23 is splined to the proximal drive sleeve 21, the proximal drive sleeve 21 can no longer rotate relative to the inner body 10. However, since the proximal drive sleeve 21 is also splined to the clutch 28, also the clutch 28 and the knob 13 can no longer rotate relative to the inner body 10.

The distally directed force applied to the knob 13 has as a consequence that the clutch 28 together with the knob 13 slightly moves in distal direction D relative to the dial sleeve 27 so that the clutch spring 25 is compressed, as already mentioned. The dog toothed engagement between the dial sleeve 27 and the clutch 28 is thereby released so that the dial sleeve 27 is no longer rotationally locked to the clutch 28. Therefore, when the knob 13 is pressed in distal direction D, the dial sleeve 27 together with the number sleeve 26 can still rotate relative to the inner body 10. When the knob 13 is now moved in distal direction D, a stop against the dial sleeve 27 forces the dial sleeve 27 to also move in distal direction D. Due to the threaded engagement of the dial sleeve 27 with the inner body 10, the dial sleeve 27 together with the number sleeve 26 moves on a helical path in distal direction D. Thereby, the numbers of the number sleeve 26 visible in the window 12 decrease.

At the same time, the clutch 28, the clickers 23, 24 and the drive sleeve 20, 21, 22 are forced to move in distal direction D (without rotation). The threaded engagement between the plunger rod 29 and the distal drive sleeve 20 forces the plunger rod 29 to rotate. A further threaded engagement between the plunger rod 29 and an inner thread of the inner body 10 may then force the plunger rod 29 to also move distally in order to push the stopper 17 inside the cartridge 16 in distal direction D for dispensing the set drug dose. Since the distal drive sleeve 20 is not rotate during dispensing, the last dose nut 30 moves together with the distal drive sleeve 20 in distal direction D without changing its position relative to the distal drive sleeve 20.

After having dispensed the set drug dose and when the knob 13 has been completely moved back into its initial position, a new drug dose may be set by again rotating the knob 13 on a helical path in proximal direction P. During this, the plunger rod 29 does not change its position. Only when dispensing a dose, the plunger rod 29 is moved in distal direction D.

As explained with respect to FIG. 1, one user interface member in form of a knob 13 is used for setting a drug dose as well as for dispensing the drug dose. However, it is also possible to use separate user interface members for setting and dispensing a drug dose.

Further examples of the drive unit 3 to be implemented with the injection device 1 can be found in WO 2014/033195 A1 or WO 2014/033197 A1 the entirety of which being incorporated herein by reference.

The drive unit 3 and in particular the drive unit housing 9 comprises a dedicated fastening structure 32, 34 as shown in FIG. 5. Here and only by way of an example the fastening structure 32, 34 is provided by the window 12 protruding radially outwardly from an outside surface of the tubular shaped drive unit housing 9.

In effect, the radially outwardly protruding window 12 comprises a window insert assembled and arranged in a respective through opening of the tubular shaped outer body 11. The fastening structure 32 comprises a first side edge and the oppositely located fastening structure 34 comprises a second side edge. Oppositely located side edges of the window 12 serve as the first and second fastening structures 32, 34 to engage with a complementary-shaped counter fastening structure e.g. on a bottom of the housing 201 of a supplementary device 200. The bottom of the housing 201 faces towards the outside surface of the drive unit housing 9 and is configured to mechanically engage with the fastening structures 32, 34.

As further illustrated in FIG. 5 and only by way of example the supplementary device 200 comprises a first fastener 202 and a second fastener 204. The fasteners 202, 204 may be implemented as a clip joint or as clip-type fasteners allowing and supporting a form fitting of the supplementary device 200 to the drive unit housing 9.

The fasteners 202, 204 and the fastening structures 32, 34 are configured to fasten and to fix the supplementary device 200 in a well-defined position or orientation relative to the drive unit housing 9. The drug delivery device 1 and the supplementary device 200 form or constitute a drug delivery system 400, which in the present example is implemented as an drug injection system.

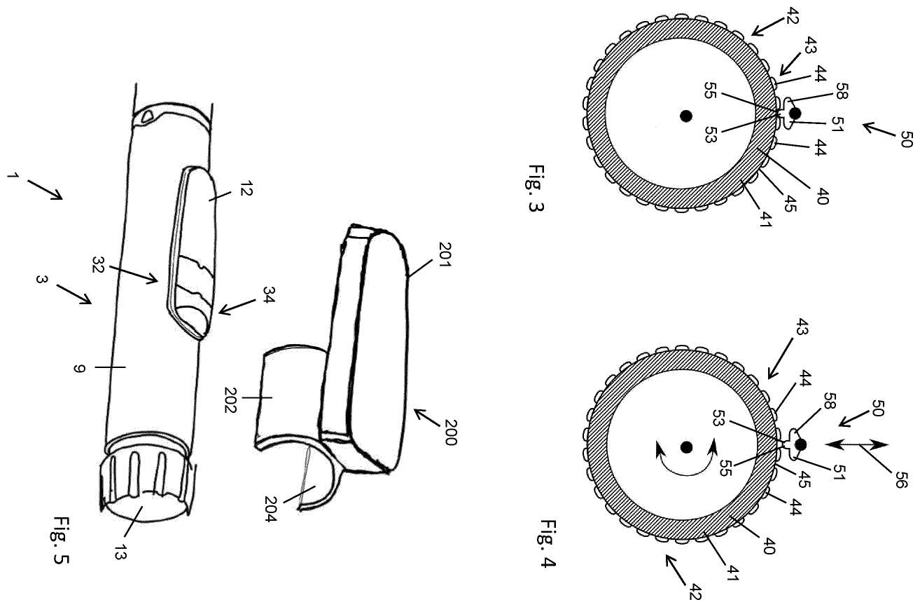

As it is particularly illustrated in FIGS. 2-4 the drive unit 3 comprises a movable drive unit component 40. In the present example the drive unit component 40 is movable during setting of a dose as well as during dispensing of a dose. The drive unit component 40 may be provided or constituted by the number sleeve 26 as described above. It is subject to a rotation, e.g. to a helical rotation relative to the inner body 10 during and/or for setting of a dose of the medicament. The drive unit 3 further comprises a locking mechanism 50. The locking mechanism 50 comprises a mechanical locking element 51. The locking element 51 is transferable between a release position as illustrated in FIGS. 2 and 4 at a locking position as illustrated in the cross-section of FIG. 3. When in the locking position the mechanical locking element 51 is in an interlocked configuration with the movable drive unit component 40. Hence, when the mechanical locking element 51 is in the locking position it serves to block and/or to impede a rotational movement and/or to block or to impede a longitudinal movement of the drive unit component 40 relative to the drive unit housing 9.

As it is further apparent from FIGS. 2-4 the mechanical locking element 51 comprises a latch 53 with a protrusion 55 to engage with a complementary shaped toothed structure 43 on the outside surface 42 of the sidewall 41 of the tubular shaped device unit component 40. The toothed structure 43 comprises a circumferential sequence of radially protruding teeth 44 and recesses 45.

In the locking configuration or blocking position as shown in FIG. 3 the latch 53, in particular a radially inwardly extending protrusion 55 of the latch 53 is in engagement with a recess 45 located between two circumferentially located consecutive teeth 44 of the toothed structure 43. In this position of the locking element 51 the device unit component 40 is rotationally locked to the locking element 51. The locking element 51 itself is pivotally supported on an inside of the drive unit housing 9. It is pivotable with regards to a pivot axis 52 as illustrated in FIG. 2. The pivot axis 52 may extend along a tangential direction or along the longitudinal direction with regards to the tubular shape of the drive unit housing 9. The pivot axis 52 and hence the mechanical locking element 51 is fixed to the drive unit housing 9. It may be fixed to the window 12 or window insert, which is fixed to the sidewall of the drive unit housing 9. In effect, the locking mechanism 50 with the locking element 51 comprises a pawl to mechanically engage with the toothed structure 43 of the device unit component 40.

The locking element 51 is transferable into a release configuration as illustrated in FIGS. 2 and 4 only or exclusively by way of an electrically controlled release mechanism 250 of the supplementary device 200. The supplementary device 200 comprises an electronic unit 270 featuring a printed circuit board 271, a processor 272, a battery 273 and an electromagnet 277, e.g. implemented as or comprising a solenoid. The electromagnet 277 forms or constitutes an electromechanical actuator 251. By applying a suitable electric current to the electromagnet 277 the actuator 251 is appropriately actuated. The actuator 251, hence the electromagnet 277 controlled by the processor 272 is operable to generate a magnetic field. The magnetic field is configured to magnetically interact with a body 58 of the locking element 51. The body 58 or at least a portion thereof comprises a ferromagnetic material or a magnetized material to magnetically interact with the magnetic field as provided or generated by the electromechanical actuator 251 or the electromagnet 277. In this way, there can be applied a magnetic force to the mechanical locking element 51 operable to lift or to move the latch 53 out of engagement from the toothed structure 43.

Transferring the mechanical locking element 51 into a release configuration or release position as shown in FIG. 4 unlocks the locking mechanism 50 and allows and supports a rotation of the device unit component 40 relative to the drive unit housing 9. Instead of a pivoting arrangement of the mechanical locking element 51 it is likewise conceivable that the mechanical locking element 51 is elastically deformable. Here, an elastically deformable part of the locking element 51 may be provided with the ferromagnetic or magnetized material so as to magnetically interact with the magnetic field as provided or generated by the electromagnet 277.

With an elastically deformable locking element 51 there may be intrinsically provided respective return forces, which in the absence of a magnetic field serve to bring or to transfer the mechanical locking element 51 into the locking configuration or blocking position. When the locking element 51 and hence the locking mechanism 50 comprises a pivotable latch 53 it is of particular benefit to have a return spring 56 as indicated in FIGS. 2 and 4 by way of which the mechanical locking element 51 is urged or biased towards the locking position. Then and in the absence of an interaction with the electrically controlled release mechanism 250 the mechanical locking element 251 is and remains in locking engagement with the drive unit component 40.

In effect and for unlocking the locking mechanism 50 it is required to appropriately mount the supplementary device 200 on the drive unit housing 9 and to generate a respective magnetic field operable to transfer the mechanical locking element 51 into the release position. Here, the supplementary device 200, in particular its electronic unit 270 may be operable to control activation of the actuator 251 for transferring the mechanical locking element 51 into the release position or unlocking position.

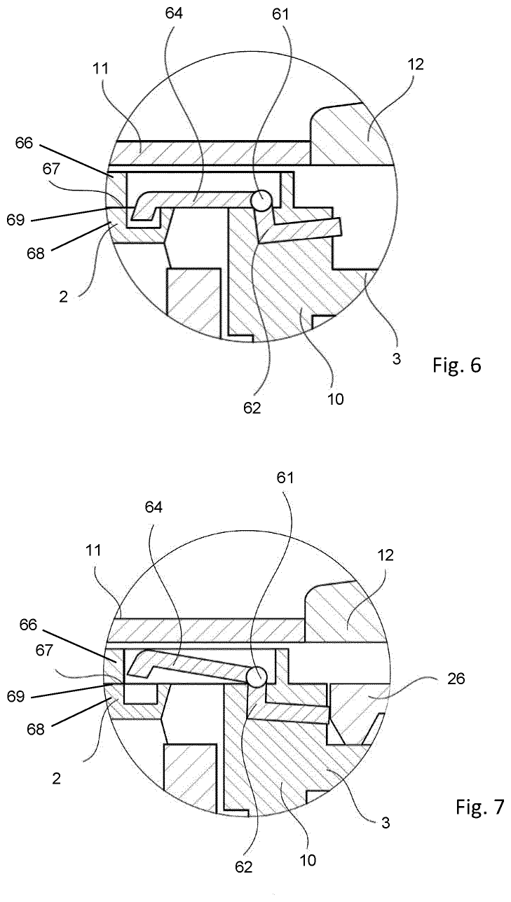

FIGS. 6 and 7 show a portion of the drive unit 3 near its distal end, which is configured to detachably fasten to the container unit. These figures indicate the functional principle of a container locking mechanism 60 configured to prevent disconnecting or separating the container unit 2 from the drive unit 3 when a drug dose is set but not fully dispensed.

One of the inner body 10 and the outer body 11 comprises an interface feature 66, e.g. in form of an inner thread 67. The reservoir unit 2, in this case the reservoir holder 15 of the drug reservoir unit 2, comprises an interface feature 68, e.g. in form of an outer thread 69. The two threads 67, 69 may be engaged and thereby establish a connection interface in form of a threaded interface via which the reservoir unit 2 is releasably connected to the drive unit 3. For releasing the connection and the connection interface, the reservoir unit 2 may have to be rotated and moved in distal direction P with respect to the body 10, 11.

In FIG. 6, however, the release of the connection interface is prevented by a locking mechanism 60 being in a locked state. The locking mechanism 60 comprises a coupling element 64 which is in a lock position in which it engages with the reservoir holder 15. The coupling element 64 comprises a coupling feature in form protrusion at its distal end which engages into a coupling feature, namely a recess or groove, of the reservoir holder 15. This engagement prevents that the reservoir unit 2 can be moved axially, optionally also rotationally, with respect to the body 10, 11 so that release of the connection interface is prevented.

The coupling element 64 is pivotably suspended in the drive unit 3 via a joint connection 61 to the inner body 10. Due to this joint connection 61, the coupling element 64 can be rotated out of the locking position of FIG. 6 into a release position shown in FIG. 7.

The coupling element 64 is an elongated element with a main section extending essentially in longitudinal direction and a further section 62 extending perpendicularly to the main section and to the rotational axis around which the coupling element 64 is rotatable. The coupling element 64 is arranged such that the number sleeve 26, which constitutes a part of the locking mechanism 60 and which moves in longitudinal direction during dose setting and dose dispensing, can, when it reaches a first position (see FIG. 7) hit the coupling element 64 radially offset of the joint connection 61 in order to exert a torque onto the coupling element 64. Due to this torque, the coupling element 64 is moved out of the lock position into the release position of FIG. 7. This happens purely mechanically via a leverage effect.

As can be seen in FIG. 7, when the number sleeve 26 is in the first position and the coupling element 64 accordingly is in the release position, the coupling element 64 is no longer engaged with the reservoir unit 2 and release of the connection interface is enabled. This state of the locking mechanism 60 is called released state. A user can now separate the reservoir unit 2 form the drive unit 3, e.g. in order to exchange the reservoir unit 2 or the drug reservoir 16. When the number sleeve 26 is moved in proximal direction P, e.g. during dose setting, the coupling element 64 automatically returns into its locking position and release of the connection interface would again be prevented.

The locking mechanism 60 described in connection with FIGS. 6 and 7 is particularly useful for preventing a user to change the reservoir unit 2 when a non-zero drug dose is currently set. As mentioned, setting a drug dose is associated with a movement of the number sleeve 26 in proximal direction P. The locking mechanism 60 may be used in any of the exemplary embodiments of a drug delivery device describe herein.

In FIGS. 9-11 there is illustrated another example of an injection device 101 provided with an electronic unit 270 at its proximal end and provided container unit 2. The device 101 of FIGS. 9-11 comprises a different type of a drive unit 103 compared to the examples if FIGS. 1-7. This particular drive unit 103 is explained in more detail e.g. in WO2004/078239A1, WO 2004/078240A1 or WO 2004/078241A1 the entirety of which being incorporated herein by reference.

The drive unit 103 comprises a number sleeve 180 that is configured to move when a dose dial 112 is turned, in order to provide a visual indication of a currently set dose. The dose dial 112 is rotated on a helical path with respect to the housing 109 when turned during setting and/or dispensing or expelling of a dose. The housing 109 may comprise a window 108, e.g. in form of an aperture through which a portion of an outside surface of the number sleeve 180 is visible.

The injection device 101 may be configured so that turning the dose dial 112 causes a mechanical click sound to provide acoustical feedback to a user. The number sleeve 180 mechanically interacts with the stopper 117 in the cartridge or drug container 116. During delivery of dose, the dose dial 112 is turned to its initial position in an axial movement, that is to say without rotation, while the number sleeve 180 is rotated to return to its initial position, e.g. to display a dose of zero units. The injection device 101 may be implemented as a disposable injection device. The cartridge holder 115 is sized and configured to accommodate the drug container 116. With its proximal end it is non-detachably fastened to the proximal housing component 109, e.g. implemented as a body of the drive unit 103. The cartridge holder 115 is to be covered by protective cap 114 detachably fastened to the cartridge holder 115 and/or to the housing 109.

A flange like support of and/or inside the housing 109 comprises a threaded axial through opening threadedly engaged with a first thread or distal thread of a piston rod 120. The distal end of the piston rod 120 comprises a bearing on which a pressure foot 123 is free to rotate with the longitudinal axis of the piston rod 120 as an axis of rotation. The pressure foot 123 is configured to axially abut against a proximally facing thrust receiving face of the stopper 117 of the container 116. During a dispensing action the piston rod 120 rotates relative to the housing 109 thereby experiencing a distally directed advancing motion relative to the housing.

The piston rod 120 is provided with a first thread 122 at or near a distal end and is further provided with a second thread 124 at its proximal end. The first thread 122 and the second thread 124 are oppositely handed. There is further provided a drive sleeve 130 having a hollow interior to receive the piston rod 120. The drive sleeve 130 comprises an inner thread threadedly engaged with the proximal thread of the piston rod 120. Moreover, the drive sleeve 130 comprises an outer threaded section 131 at its distal end. The threaded section 131 is axially confined between a distal flange portion 132 and another flange portion 133, e.g., a proximal flange portion located at a predefined axial distance from the distal flange portion 132. Between the two flange portions 132, 133 there is provided a last dose limiter 135 in form of a semi-circular nut having an internal thread mating the threaded section 131 of the drive sleeve 130.

The last dose limiter 135 further comprises a radial recess or protrusion at its outer circumference to engage with a complementary-shaped recess or protrusion at an inside of the sidewall of the housing 109. In this way the last dose limiter 135 is splined to the housing 109. A rotation of the drive sleeve 130 in a dose incrementing direction or clockwise direction during consecutive dose setting procedures leads to an accumulative axial displacement of the last dose limiter 135 relative to the drive sleeve 130.

There is further provided an annular spring 136 that is in axial abutment with a proximally facing surface of the flange portion 133. Moreover, there is provided a tubular-shaped clutch 170. At a first end the clutch 170 is provided with a series of circumferentially directed saw teeth. Towards a second opposite end of the clutch 170 there is located a radially inwardly directed flange.

Furthermore, there is provided a dose dial sleeve also denoted as number sleeve 180. The number sleeve 180 is provided outside of the spring 136 and the clutch 170 and is located radially inward of the housing 109. A helical groove 181 is provided about an outer surface of the number sleeve 180. The housing 109 is provided with a dosage window through which a part of the outer surface of the number sleeve 180 can be visually inspected. The housing 109 is further provided with a helical rib 111 at an inside sidewall portion of an insert piece 110, which helical rib 111 is to be seated in the helical groove 181 of the number sleeve 180. The tubular shaped insert piece 110 is inserted into the proximal end of the housing 109. It is rotationally and axially fixed to the housing 109. There are provided first and second stops on the housing 109 to limit a dose setting procedure during which the number sleeve 180 is rotated in a helical motion relative to the housing 109.

The dose dial 112 in form of a dose dial grip is disposed about an outer surface of the proximal end of the number sleeve 180. An outer diameter of the dose dial 112 typically corresponds to and matches with the outer diameter of the housing 109 and may form or belong to the housing 109. The dose dial 112 may be rotationally secured to the number sleeve 180 to prevent relative movement therebetween. The dose dial 112 is provided with a central opening.

The trigger 113, also denoted as dose button is substantially T-shaped. It is provided at a proximal end of the injection device 1. It may form or constitute a proximal end of the housing 109. A shaft 119 of the trigger 113 extends through the opening in the dose dial 112, through an inner diameter of extensions of the drive sleeve 130 and into a receiving recess at the proximal end of the piston rod 120. The shaft 119 is retained for limited axial movement in the drive sleeve 130 and against rotation with respect thereto. A head of the trigger 113 is generally circular. A trigger side wall or skirt extends from a periphery of the head and is further adapted to be seated in a proximally accessible annular recess of the dose dial 112.

To dial a dose a user rotates the dose dial 112. With the spring 136 and the clutch 170 engaged, the drive sleeve 130, the spring 136, the clutch 170 and the number sleeve 180 rotate with the dose dial 112. Torque is transmitted through saw teeth between the spring 136 and the clutch 170. The helical groove 181 on the number sleeve 136 and a helical groove in the drive sleeve 130 have the same lead. This allows the number sleeve 180 to extend from the housing 109 and the drive sleeve 130 to climb the piston rod 120 at the same rate. At a limit of travel a radial stop on the number sleeve 180 engages either with a first stop or a second stop provided on the housing 109 to prevent further movement in a first sense of rotation, e.g. in a dose incrementing direction. Rotation of the piston rod 120 is prevented due to the opposing directions of the overall and driven threads on the piston rod 120.

The last dose limiter 135 keyed to the housing 109 is advanced along the threaded section 131 by the rotation of the drive sleeve 130.

During dialing or setting of a dose a ratchet mechanism allows and supports a rotation of the number sleeve 180 relative to the clutch 170 along a second sense of rotation, which rotation is accompanied by a regular clicking of a flexible arm of the clutch 170. An angular momentum applied to the number sleeve 180 along the first sense of rotation is unalterably transferred to the clutch 170. Here, the mutually corresponding ratchet features of the ratchet mechanism provide a torque transmission from the number sleeve 180 to the clutch 170.

When the desired dose has been dialed the user may simply dispense the set dose by depressing the trigger 113. This displaces the clutch 170 axially with respect to the number sleeve 180 causing dog teeth thereof to disengage. However, the clutch 170 remains keyed in rotation to the drive sleeve 130. The number sleeve 180 and the dose dial 112 are now free to rotate in accordance with the helical groove 181.

The axial movement deforms the flexible arm of the spring 136 to ensure the saw teeth cannot be overhauled during dispense. This prevents the drive sleeve 130 from rotating with respect to the housing 109 though it is still free to move axially with respect thereto. The deformation is subsequently used to urge the spring 136 and the clutch 170 back along the drive sleeve 130 to restore the connection between the clutch 170 and the number sleeve 180 when the distally directed dispensing pressure is removed from the trigger 113.

The longitudinal axial movement of the drive sleeve 130 causes the piston rod 120 to rotate through the through opening of the support of the housing 109, thereby to advance the stopper 17 in the container 16. Once the dialed dose has been dispensed, the number sleeve 180 is prevented from further rotation by contact of at least one stop extending from the dose dial 112 with at least one corresponding stop of the housing 109. A zero dose position may be determined by the abutment of one of axially extending edges or stops of the number sleeve 180 with at least one or several corresponding stops of the housing 109.

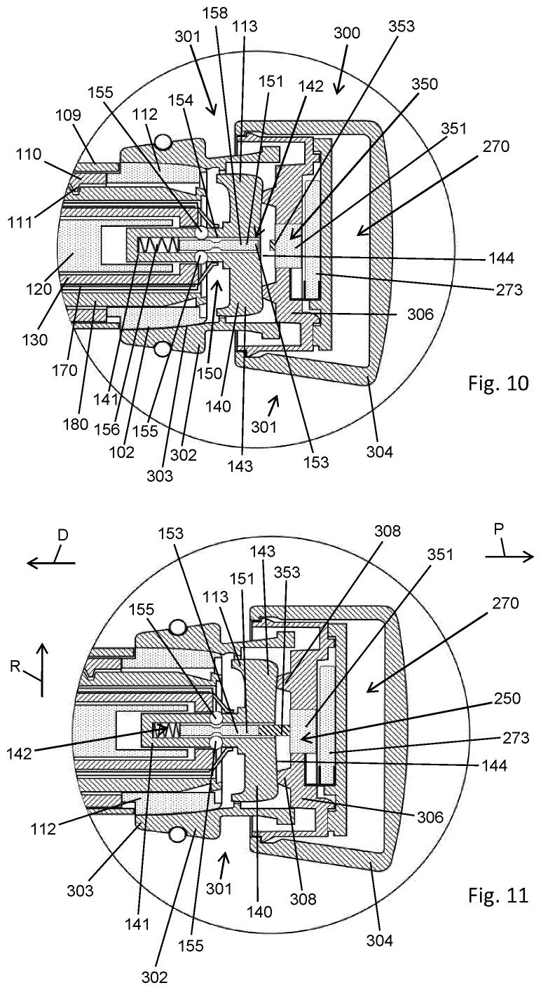

With the further example of FIGS. 9-11 the supplementary device 300 is configured for attachment to the dose dial 112 provided at the proximal end of the drug delivery device 101. As shown in FIGS. 10 and 11 the supplementary device 300 comprises a multicomponent housing 301. Here, a first housing component 302 is configured for fastening to the dose dial 112. When the supplementary device 300 is appropriately fastened by way of its fastener 303, e.g. to a fastening structure 102 provided on the outside circumference of the dose dial 112, there can be formed a friction fit with the outside surface of the dose dial 112. Then a release mechanism 350 of the supplementary device 300 may be appropriately aligned with a bore 142 of a drive unit component 140.

A second housing component 304 is longitudinally movable relative to the first housing component 302. While the first housing component 302 is and remains fixed to the dose dial 112 the second housing component 304 is mechanically engaged with a carrier 306 featuring at least one distally facing abutment or protrusion 308 by way of which the carrier 306 and hence the second housing portion 304 is configured to get into longitudinal abutment with an outside facing end face 144 of the trigger 113. Here, the trigger 130 is implemented as the drive unit component 140 being movable relative to the drive unit housing 109 for dispensing of the dose of the medicament.

Here, the drive unit 103 of the injection device 101 comprises a different kind of a locking mechanism 150, which is integrated or implemented inside the drive unit component 140 presently implemented as the trigger 113 or trigger button.

As shown in FIGS. 10 and 11 the locking mechanism 150 comprises a mechanical locking element 151 implemented as a locking bolt 153 movable in longitudinal direction inside the bore 142 of the drive unit component 140. The drive unit component 140 comprises a radially widened flange portion 143 and a distally extending shaft featuring a somewhat tubular-shaped sidewall 141. The bore 142 forms or comprises a cup-shaped receptacle open towards the proximal direction P. In the bottom of the bore 142 there is arranged a return spring 156 which is in longitudinal or axial engagement with a distal end or with a distally facing abutment face of the locking element 151.

The locking element 151 comprises body 158 with a cross-section or diameter that matches with the respective cross-section or diameter of the bore 142. The size of the bore 142 is smaller than a fingertip. It is also smaller than a conventionally tipped object, such as a ballpoint pen. When the supplementary device 300 is appropriately fastened by way of its fastener 303 to the outside circumference of the dose dial 112, thereby forming a friction fit with the outside surface of the dose dial 112, the release mechanism 350 of the supplementary device 300 may be appropriately aligned with the bore 142.

Here, the release mechanism 350 comprises an electromechanically implemented actuator 351 comprising a pin 353 movable in longitudinal direction A. The actuator 351 is operable and/or actuatable by the electronic unit 270. When the supplementary device 300 is correctly assembled to the dose dial 112 and hence to the proximal end of the drive unit housing 109 the pin 353 is in longitudinal alignment with the bore 142. The cross-section or diameter of the pin 353 is sized to enter the bore 142 and is further operable to apply a distally directed force effect onto the locking element 151. With the activated actuator 351 and hence with a distally projected pin 353 the locking element 151 is urged in distal direction D inside the bore 142 against the action of the return spring 156. The movement of the pin 353 is controllable by the electronic units 270 as will be described further below.

As further illustrated in FIGS. 10 and 11 the drive unit component 140 further comprises at least one or a plurality of retaining elements 155. With the presently illustrated example the retaining elements 155 each comprise a spherical geometry. The retaining elements 155 are located in a portion of the sidewall 141 of the drive unit component 140. The retaining elements 155 are movable in radial direction R from a retaining position as shown in FIG. 10 towards a release position as illustrated in FIG. 11. In the retaining position the retaining elements 155 protrude at least slightly radially outwardly from the sidewall 141 of the drive unit component 140. Moreover, the retaining elements 155 are axially fixed or axially confined in or to the sidewall 141.

The retaining elements 155 axially or longitudinal engage with a respective surface or abutment of the drive sleeve 130, e.g. provided at a proximal end of the drive sleeve 130. The drive sleeve 130 serves as a further drive unit component, which is longitudinally fixed to the drive unit housing 109, as long as the clutch 170 is engaged. Disengagement of the clutch 170 involves a distally directed depression or movement of the trigger 113 and hence of the drive unit component 140.

Such a distally directed movement is effectively blocked by the retaining elements 155 as long as they are in a longitudinal engagement or longitudinal abutment with the drive sleeve 130. For unlocking the locking mechanism 150, the locking element 151 is pushed and hence the locking bolt 153 in distal direction D. As shown in FIG. 10, the locking bolt 153, comprises a radial recess 154, which is in alignment with the retaining elements 155 when the locking element 151 reaches the release position relative to the drive unit component 140. The recess 154, e.g. implemented as an annular-shaped groove on the outside surface of the locking bolt 153, is sized to receive at least a portion of the retaining element(s) 155.

As shown in FIG. 11 the retaining elements 155 may enter the recess 154 and may simultaneously get out of engagement from the drive sleeve 130. Then and by a radially inwardly directed movement of the retaining element(s) 155, which is possible due to the longitudinal alignment of the recess 154 with the retaining element(s) 155 the retaining element(s) 155 get out of engagement from the drive sleeve 130 and do therefore no longer block a distally directed movement of the drive unit component 140 relative to the drive unit housing 109.

By deactivating the release mechanism 350, hence by retracting the pin 353 towards the proximal direction D the blocking element 151 is subject to a respective proximally directed sliding movement under the action of the return spring 156. Then, the retaining elements 155 will be urged radially outwardly due to the beveled shape of the radial 154 as provided on the locking bolt 153.

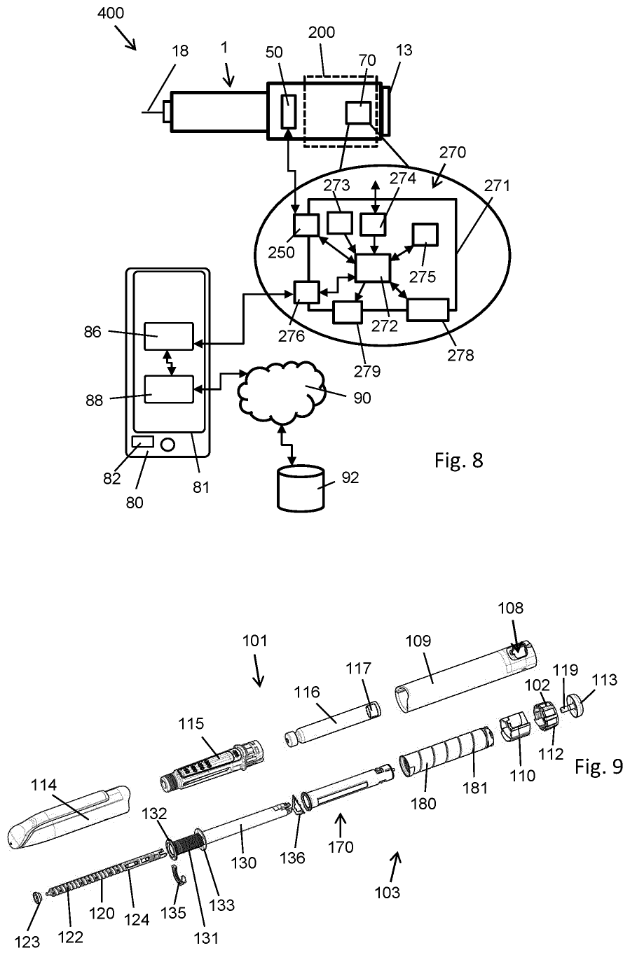

FIG. 8 is a schematic block diagram of a supplementary device 200, 300 mounted to the drug delivery device 1, 101 and being operable to modify or to control the locking mechanism 50, 150 of the injection device.

The supplementary device 200 is inter alia equipped with a printed circuit board (PCB) 271. There is provided a processor 272 on the PCB. The processor 272 may for instance be a microprocessor, a Digital Signal Processor (DSP), Application Specific Integrated Circuit (ASIC), Field Programmable Gate Array (FPGA) or the like. Processor 272 is configured and operable to execute program code (e.g. software or firmware) stored in a storage 275. The storage 275 may comprise a program memory and may use a main memory, for instance to store intermediate results. The storage 275 may also be used to store a logbook on performed ejections/injections. The storage 275, hence the program memory may for instance be a Read-Only Memory (ROM), and main memory may for instance be a Random Access Memory (RAM).

With some examples, processor 272 interacts with an input, e.g. in form of one or a plurality of buttons or actuators by way of which the supplementary device 200, 300 may for instance be turned on and off. A further button may be implemented as a communications button. The further button may be used to trigger establishment of a communication link or data connection to an external electronic device 80, which is e.g. implemented as a smart phone. Still another button may be implemented as a confirmation button or OK button. This button may be used to acknowledge information presented to a user of the supplementary device 200, 300. The buttons may be any suitable form of user input transducers, for instance mechanical switches, capacitive sensors or other touch sensors.

The processor 272 may control a display 278 or display unit, which is presently embodied as a Liquid Crystal Display (LCD). The optional display 278 is operable to display information to a user of the supplementary device 200, 300, for instance on present settings of injection device 1, 101, or on a next injection to be conducted. The display 278 or display unit may also be embodied as a touch-screen display, for instance to receive user input.

The processor 272 may be further operationally coupled to a sensor 274. The sensor 274 may be implemented as an optical sensor, embodied as an Optical Character Recognition (OCR) reader, that is capable of capturing images of the window 12 or dosage display, in which a currently selected dose is displayed (by way of numbers printed on the sleeve 26 contained in injection device 1, which numbers are visible through the window 12 or dosage display). The OCR reader may be further capable of recognizing characters (e.g. numbers) from the captured image and to provide this information to the processor 272.

Alternatively, the sensor 274 of the supplementary device 200 may only be an optical sensor, e.g. a camera, for capturing images and providing information on the captured images to processor 272. Then, the processor 272 might by operable to perform OCR on the captured images.

The processor 272 may also control light-sources such as light emitting diodes to illuminate the window 12, in which a currently selected dose is displayed. A diffuser may be used in front of the light-sources, for instance a diffuser made from a piece of acrylic glass. Furthermore, the optical sensor 274 may comprise a lens (e.g. an aspheric lens) leading to a magnification (e.g. a magnification of more than 3:1).

The sensor 274 may be also implemented as an acoustic sensor, which is configured to sense sounds produced by the injection device 1, 101. Such sounds may for instance occur when a dose is dialed, e.g. by turning the dose dial 112 or dose button 13, and/or when a dose is ejected/injected by the dose button 13 or trigger 113, and/or when a prime shot is performed. These actions are mechanically similar but nevertheless sound differently (this may also be the case for electronic sounds that indicate these actions). Here, either the sensor 274 and/or processor 272 may be configured to differentiate these different sounds, for instance to be able to safely recognize that an injection has taken place (rather than a prime shot only).

The processor 272 may further control a signal generator 279, e.g. implemented as an acoustic signal generator which is configured to produce acoustical signals that may for instance be related to the operating status of injection device 1, 101, for instance as a feedback to the user. For example, an acoustical signal may be launched by the signal generator 279 as a reminder for the next dose to be injected or as a warning signal, for instance in case of misuse. Acoustical signal generator may for instance be embodied as a buzzer or loudspeaker. In addition to or as an alternative to acoustical signal generator 279, also a haptic signal generator (not shown) may be used to provide haptic feedback, for instance by way of vibration.

The processor 272 further controls a communication interface 276, e.g. implemented as a wireless unit or as a wireless transceiver. The communication interface 276 is configured to transmit and/or to receive information to or from another device in a wireless fashion. Such transmission may for instance be based on radio transmission or optical transmission. Typically the communication interface 276 is implemented pursuant to commercially available wireless communication standards, such as Bluetooth, Wi-Fi, UWB or NFC.

Alternatively, the communication interface 276 may comprise a wired unit configured to transmit and/or receive information to/from another device in a wire-bound fashion, for instance via a cable or fibre connection. When data is transmitted, the units of the data (values) transferred may be explicitly or implicitly defined. For instance, in case of an insulin dose, always International Units (IU) may be used, or otherwise, the used unit may be transferred explicitly, for instance in coded form.

The processor 272 may be operable to receive an input from a pen detection switch (not illustrated), which is operable to detect whether the pen 1 or drug delivery is present, e.g., to detect whether the supplementary device 200 is correctly coupled to the injection device 1, 101. A battery 273 powers the processor 272 and other components of the electronica unit 270.

The supplementary device 200, 300 is thus capable of determining information related to a condition and/or use of injection device 1, 101. This information may be displayed on the display 21 for use by the user of the device. The information may be either processed by supplementary device 200, 300 itself, or may at least partially be provided to an external electronic device 80 for further data processing.

Generally, and by way of the sensor 274 information regarding the size of the dose currently set, dialed or dispensed can be obtained and can be stored in a local storage 275. Data repeatedly captured or obtained during repeated and subsequent use of the injection device 1, 101 can be stored in the storage 275. Such injection-related data stored in the storage 275 can be synchronized, e.g. by the communication interface 276 communicating with the external electronic device 80 and/or with an external database 92, e.g. hosted or provided by a healthcare provider.

Typically, the external electronic device 80 is implemented as a smart phone, as a smart watch or as a tablet computer. It may comprise a wide range communication interface 88 to establish a communication link to the external database 92 via a communication network 90. Typically, the external electronic device 80 is a mobile device or wearable device. It comprises a display 81 and/or a speaker 82 to communicate with a user.

The external electronic device 80 may be wirelessly paired to the electronic unit 70. Upon successful pairing a respective confirmation may be provided to a user, e.g. via the display 81 and/or via the speaker 82 of the external electronic device 80. Upon transferring of data or information between the electronic unit 70 and the external electronic device 80 a visual and/or audible confirmation may be provided to the user, e.g. by the electronic unit 70 and/or by the external electronic device 80.

The processor 272 may be configured to implement an automatic re-locking, e.g. after termination of each dispensing or in injecting procedure. Moreover, and based on signals obtainable from the sensor 274 the processor 272 may be provided with information being indicative of the remaining filling level of the medicament container 16. In this way and when the sensor 274 provides respective sensor signals that the medicament container 16 is substantially depleted or empty the processor 272 may control and/or actuate the release mechanism 250, 350 and thus the actuator 251, 351 to transfer the mechanical locking element 51, 151 into the locking position.

With further examples the electrically controlled release mechanism 250, 350 of the supplementary device 200, 300 may be controlled by the processor 272 on the basis of an authentication routine conducted by the supplementary device 200, 300 and/or conducted by the external electronic device 80. This may particularly apply with reusable injection devices 1, 101 having a common drive unit 3, 103 but distinguish by their container unit 2 and/or by the medicament stored in the medicament container 16, 116.

Here, a particular container unit 2 equipped with a medicament container 16, 116 may be visually or electronically encoded by a respective coding feature, e.g. implemented as a barcode, as a QR code or in form of an electronically readable code, e.g. provided by RFID chip. At least one of the supplementary device 200, 300 and the external electronic device 80 may be operable to capture and/or to read or to extract the coding feature of the container unit 2. Based on the captured coding feature the processor 272 may be operable to activate or to deactivate the release mechanism 250, 350. If a wrong container unit 2 should be paired and/or connected to the drive unit 3, the coding feature captured and/or processed by the processor 272 will be inoperable to activate the release mechanism 250, 350 for switching the mechanical locking element into the release position. Then, the locking mechanism 50, 150 may remain in the locked position or locked configuration. Misuse or unintended use of the drive unit 3, 103 is then effectively prevented.

The electronic unit 270, in particular its processor 272 may be operable to activate the release mechanism 250, 350 only when a correct coding feature is detected. The evaluation of a correct coding feature may be conducted by the processor 272 and/or by a processor of the external electronic device 80. Here, approval of an authentication to unlock the locking mechanism 50, 150 may be also conducted by the external electronic device.

### REFERENCE NUMBERS

1 drug delivery device
2 container unit
3 drive unit
4 distal end
5 proximal end
9 drive unit housing
10 inner body
11 outer body
12 window
13 dose button
14 protective cap
15 cartridge holder
16 drug container
17 stopper
18 injection needle
20 distal drive sleeve
21 proximal drive sleeve
22 drive sleeve coupler
23 distal clicker
24 proximal clicker
25 clutch spring
26 number sleeve
27 dial sleeve
28 clutch
29 plunger rod
30 last dose nut
32 fastening structure
34 fastening structure
40 device unit component
41 sidewall
42 outside surface
43 toothed structure
44 tooth
45 recess
50 locking mechanism
51 locking element
52 pivot axis
53 latch
55 protrusion
56 return spring
58 body
60 container locking mechanism
61 joint connection
62 section
64 coupling element
66 interface feature
67 thread
68 interface feature
69 thread
80 external electronic device
81 display
82 speaker
86 communication interface
88 communication interface
90 network
92 database
101 drug delivery device
102 fastening structure
103 drive unit
108 window
109 drive unit housing
110 insert piece
111 rib
112 dose dial
113 trigger
114 protective cap
115 cartridge holder
116 medicament container
117 stopper
119 shaft
120 piston rod
121 bearing
122 first thread
123 pressure foot
124 second thread
130 drive sleeve
131 threaded section
132 flange
133 flange
135 last dose limiter
136 spring
140 drive unit component
141 sidewall
142 bore
143 flange portion
144 end face
150 locking mechanism
151 locking element
153 locking bolt
154 recess
155 retaining element
156 return spring
158 body
170 clutch
180 number sleeve
181 groove
200 supplementary device
201 housing
202 fastener
204 fastener
250 release mechanism
251 actuator
270 electronic unit
271 printed circuit board
272 processor
273 battery
274 sensor
275 storage
276 communication interface
277 electromagnet
278 display
279 signal generator
300 supplementary device
301 housing
302 housing portion
303 fastener
304 housing portion
306 carrier
308 protrusion
350 release mechanism
351 actuator
353 pin
400 drug delivery system

1-17. (canceled)

18. A drive unit of a drug delivery device configured for setting and dispensing of a dose of a medicament, the drive unit comprising:
a drive unit housing extending along a longitudinal axis;
a drive unit component movably arranged in or on the drive unit housing for at least one of setting of the dose and dispensing of the dose of the medicament; and
a locking mechanism comprising a mechanical locking element transferable between a release position and a locking position, wherein the mechanical locking element is configured to mechanically engage with the drive unit component when in the locking position to block or to impede at least one of setting of the dose and dispensing of the dose of the medicament, and wherein the mechanical locking element is exclusively transferable from the locking position into the release position through interaction with an electrically controlled release mechanism of a supplementary device, wherein the supplementary device is configured for attachment to the drive unit housing.

19. The drive unit according to claim 18, wherein the mechanical locking element is movably arranged inside the drive unit housing.

20. The drive unit according to claim 19, wherein the mechanical locking element is concealed by the drive unit housing.

21. The drive unit according to claim 18, wherein the mechanical locking element is mechanically inaccessible from outside the drive unit housing.

22. The drive unit according to claim 18, wherein the drive unit housing comprises a recess or an access opening in-line with the mechanical locking element.

23. The drive unit according to claim 18, wherein the mechanical locking element comprises a body with at least one of a ferromagnetic material and a magnetized material to magnetically interact with a release mechanism of the supplementary device.

24. The drive unit according to claim 18, wherein:
the drive unit housing comprises a fastening structure configured to detachably engage with a complementary shaped fastener of the supplementary device, and
the mechanical locking element is aligned with an actuator of a release mechanism of the supplementary device when the fastening structure is correctly engaged with the complementary shaped fastener of the supplementary device.

25. The drive unit according to claim 18, wherein:
the mechanical locking element is movably fastened to one of the drive unit component and the drive unit housing, and
the mechanical locking element is configured to engage with the other one of the drive unit component and the drive unit housing when transferred from the release position into the locking position.

26. The drive unit according to claim 18, wherein:
the drive unit component is rotatable with regard to the longitudinal axis for at least one of setting of the dose and dispensing of the dose,
the drive unit component comprises a tubular-shaped sidewall with a toothed structure, and
the mechanical locking element comprises a latch movable, pivotable, or deformable in a radial direction to engage with the toothed structure.

27. The drive unit according to claim 18, wherein:
the drive unit component is longitudinally displaceable for at least one of setting of the dose and dispensing of the dose, and
the locking mechanism comprises a retaining element longitudinally secured to the drive unit component relative to the longitudinal axis and movable in radial direction relative to the longitudinal axis between a retaining position and a release position.

28. The drive unit according to claim 27, wherein:
the mechanical locking element is movably supported in or on the drive unit component and is mechanically engaged with the retaining element, and
the mechanical locking element and the retaining element are configured such that a longitudinal movement of the mechanical locking element from the locking position towards the release position causes a movement of the retaining element from the retaining position towards the release position.

29. The drive unit according to claim 18, wherein:
the drive unit component is movable from an initial position towards an end position for at least one of setting of the dose and dispensing of the dose, and
when in the locking position, the mechanical locking element is operable to fix the drive unit component in the initial position.

30. The drive unit according to claim 18, wherein the locking mechanism comprises a return spring engaged with the mechanical locking element and configured to transfer the mechanical locking element from the release position into the locking position.

31. A supplementary device for use with a drive unit of a drug delivery device configured for setting and dispensing of a dose of a medicament, the drive unit comprising:
a drive unit housing extending along a longitudinal axis;
a drive unit component movably arranged in or on the drive unit housing for at least one of setting of the dose and dispensing of the dose of the medicament; and
a locking mechanism comprising a mechanical locking element transferable between a release position and a locking position, wherein the mechanical locking element is configured to mechanically engage with the drive unit component when in the locking position to impede at least one of setting of the dose and dispensing of the dose of the medicament, and wherein the mechanical locking element is exclusively transferable from the locking position into the release position through interaction with a release mechanism of the supplementary device, the supplementary device comprising:
a housing configured for attachment to the drive unit,
an electronic unit arranged inside the housing,
the release mechanism comprising an electromagnetic actuator electrically controlled by the electronic unit and operable to transfer the mechanical locking element of the locking mechanism of the drive unit into the release position.

32. The supplementary device according to claim 31, wherein the electromagnetic actuator comprises one of:
an electromagnet operable to magnetically interact with the mechanical locking element and
a movable pin to mechanically engage with the mechanical locking element.

33. A drug delivery system comprising:
a drive unit of a drug delivery device configured for setting and dispensing of a dose of a medicament, the drive unit comprising:
a drive unit housing extending along a longitudinal axis;
a drive unit component movably arranged in or on the drive unit housing for at least one of setting of the dose and dispensing of the dose of the medicament; and
a locking mechanism comprising a mechanical locking element transferable between a release position and a locking position, wherein the mechanical locking element is configured to mechanically engage with the drive unit component when in the locking position to block or to impede at least one of setting of the dose and dispensing of the dose of the medicament; and
a supplementary device for use with the drive unit, the supplementary device comprising:
a housing configured for attachment to the drive unit,
an electronic unit arranged inside the housing,
a release mechanism comprising an electromagnetic actuator electrically controlled by the electronic unit and operable to transfer the mechanical locking element into the release position.

34. The drug delivery system according to claim 33, wherein the electromagnetic actuator comprises one of:
an electromagnet operable to magnetically interact with the mechanical locking element; and
a movable pin to mechanically engage with the mechanical locking element.

35. The drug delivery system of claim 33, wherein the mechanical locking element comprises a body with at least one of a ferromagnetic material and a magnetized material to magnetically interact with the release mechanism of the supplementary device.

36. The drug delivery system of claim 33, wherein the drive unit housing comprises a fastening structure configured to detachably engage with a complementary shaped fastener of the supplementary device, and
wherein the mechanical locking element is aligned with the electromagnetic actuator of the release mechanism of the supplementary device when the fastening structure is correctly engaged with the complementary shaped fastener of the supplementary device.

37. The drug delivery system of claim 33, further comprising a container unit configured to accommodate a medicament container and configured to fasten to the drive unit housing, wherein the drive unit is configured to operably engage with the medicament container to withdraw or to expel a dose of the medicament from the medicament container.
